(19) United States
(12) Patent Application Publication
(10) Pub. No.: US 2013/0096482 A1
(43) Pub. Date: Apr. 18, 2013

(54) ALTERNATE GEOMETRY STYLET FOR VENTRICULAR SHUNT CATHETER PLACEMENT

(71) Applicants: William Jeffrey Bertrand, Ventura, CA (US); Robert C. Leonard, Ventura, CA (US)

(72) Inventors: William Jeffrey Bertrand, Ventura, CA (US); Robert C. Leonard, Ventura, CA (US)

(73) Assignee: MEDTRONIC XOMED, INC., Jacksonville, FL (US)

(21) Appl. No.: 13/276,155

(22) Filed: Oct. 18, 2011

(51) Int. Cl. A61M 1/00; B23P 17/04

(52) U.S. Cl. 604/8; 29/428

(57) ABSTRACT

A stylet having a non-round cross-sectional shape which is specifically adapted to reduce the adhesion or “stickiness” of contact between the stylet and the interior surface of the lumen of an elastomeric catheter through which it extends. The stylet may be “pre-loaded” into the catheter.

### BACKGROUND

Conventional stylets in commercial use for ventricular shunt catheter placement are circular in cross section. Non-circular cross-sections have been disclosed but not described in sufficient detail to enable their successful commercialization. The use of those with circular cross-sections, or any geometry that is complementary to the lumen geometry, can in some cases result in large areas of surface contact between the outer surface of the stylet and the inside surface of the ventricular catheter (which typically also has a circular cross-section). As these catheters are generally made from silicon elastomer, some adhesion between the catheter and stylet can develop due to the inherent “tackiness” of most silicone elastomer materials.

In ventricular shunt applications, the stylet is moved within the catheter axially (in the proximal or distal direction); rotation (sometimes known as “torquing”) the stylet around its own axis is generally not required or performed. During such axial motion, the adhesion manifests itself as friction that resists the axial motion and therefore may complicate the maintenance of accurate placement of the tip of the catheter; this is particularly a problem when the stylet is withdrawn, as it may lead to loss of the accurate placement of the tip of the catheter by use of the stylet at the outset.

### SUMMARY

In general terms, an improved stylet exhibits reduced adhesion or friction when in contact with silicone materials in the setting described above. The stylet is manufactured from non-circular cross-section wire, e.g., wire having cross-sectional geometries that are generally triangular, square, pentagonal, hexagonal, octagonal, and the like; and such non-circular geometries are further defined as being outer surfaces which define at least some additional geometric features such as rounded faces (either concave or convex), rounded corner surfaces, or a combination of both.

In one embodiment, a stylet comprises an elongate stylet body having a proximal end, a distal end, and an outer surface comprising at least three faces. The portion having at least three faces may be the entire length of the stylet, or only that distal portion of the length which is within a catheter having a lumen with a circular cross-section. In the latter case, it is preferred that the proximal portion of the stylet have a circular cross-section, so that the “feel” of the stylet in the hand of the surgeon is not changed.

In another embodiment, a method comprises removing a stylet made of a rigid material from a catheter made of an elastomeric material. The stylet comprises an elongate stylet body having a proximal end, a distal end and an outer surface comprising at least three faces at least for a portion of its length (for example, only that distal portion of the length which is within a catheter having a lumen with a circular cross-section). The method comprises guiding the catheter loaded with the stylet to a desired target; and removing the stylet from the catheter.

In another embodiment, another method comprises manufacturing a stylet to be sufficiently rigid to be easily removed from an elastomeric catheter. The method comprises providing the stylet with an elongate stylet body having a proximal end, a distal end and an outer surface comprising at least three faces at least for a portion of its length.

Other embodiments and variations are possible beyond those described in this Summary section, and therefore nothing in this Summary section should be taken as expressing a requirement applicable to any particular commercial embodiment.

### BRIEF DESCRIPTION OF THE DRAWINGS

FIG. 1 is a schematic illustration of a portion of a stylet, indicating the direction of a transverse cross-sectional view as A-A, and the direction of a side view as B-B.

FIGS. 2-5 are transverse cross-sectional views of various alternative embodiments of a stylet, taken along the line A-A of FIG. 1.

FIGS. 6 and 7 are schematic cross-sectional views illustrating the fit of conventional and non-conventional stylets within a lumen.

### DETAILED DESCRIPTION

A very common practice in interventional medical procedures involving a catheter or other elongated object is to include some kind of stiffening member or stylet within the object. This lends a degree of temporary reduction in the flexibility of the catheter so that it may be more easily introduced or guided to its desired location within the patient. Once that is completed, the stylet may be removed. It is common to provide the catheter to the surgical site with the stylet already inserted, or “preloaded” for use.

For a variety of reasons, including the need to improve the ability of such catheters to be guided in place (often over a convoluted path), “soft” (low durometer) materials are commonly used in the construction of catheters. A common measurement scale is Shore hardness, of which there are various types (identified by different letter combinations) and a value scale of 0-100 for each type, all defined by published standards. In interventional neurological and neurosurgical applications, such as ventricular catheters, a typical durometer value for a suitable silicone material would be approximately 50 to 65 on the A scale.

Stylets are typically polished stainless steel wires having constant cylindrical cross-sections and smooth outer surfaces. Nonetheless, the softness of catheter materials leads to high amounts of friction that make it difficult to remove the stylet. It is even possible that the catheter will be moved from its desired location, or damaged, or both. Particularly in the delicate context of neurosurgery, neither is desirable.

One approach is to coat the stylet, for example with PTFE or another lubricious coating. Another is to modify the material of the catheter to reduce friction. Another approach is to modify the stylet cross-section. Yet another is to provide the stylet with some type of surface treatment. An example of surface treatment is the approach taken in US Published Patent Application 2008/0103448. The stylet is required to have a circular cross-section (the application disparages non-circular cross-sections as having unsatisfactory “feel”), and the stylet surface is roughened to a specified degree, e.g., peak heights>30 micrometer.

As suggested by the disparagement noted above, any change to the “feel” of a catheter/stylet combination may render a design unsuitable in practice, as “feel” is a very important design consideration because of the precision and time demands of the tasks involved.

The stylets disclosed here are characterized by non-circular cross-sections and further by other geometric features which reduce the amount of contact area between the stylet and the inner diameter of the catheter, but without a loss of satisfactory “feel” or other performance measures.

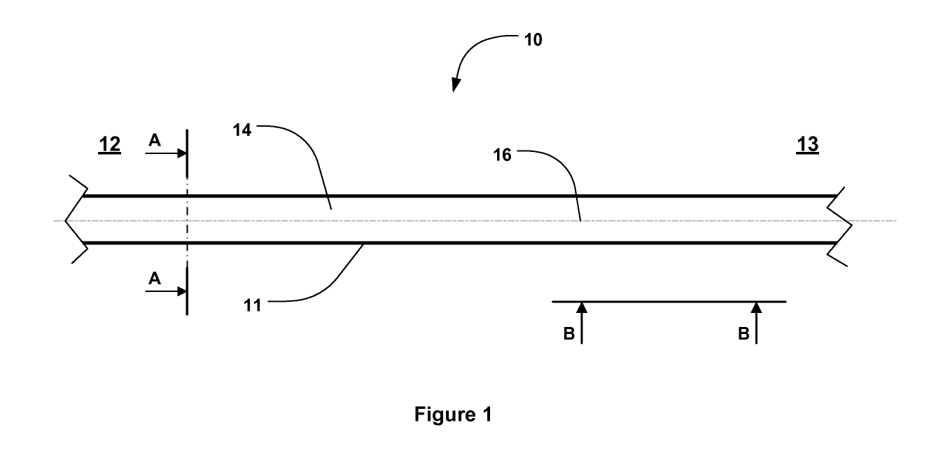

As generally illustrated in FIG. 1, a stylet 10 (for clarity, only a portion of which is shown) comprises an elongate body 11 extending between the proximal and distal directions 12, 13 and having an outer surface 14. For clarity and simplicity, FIG. 1 omits shading and contour lines that would suggest the view of the stylet taken in the longitudinal direction (indicated as B-B) or toward the longitudinal axis 16. The stylet 10 may be solid or hollow and thus is only schematically depicted as solid in the Figures.

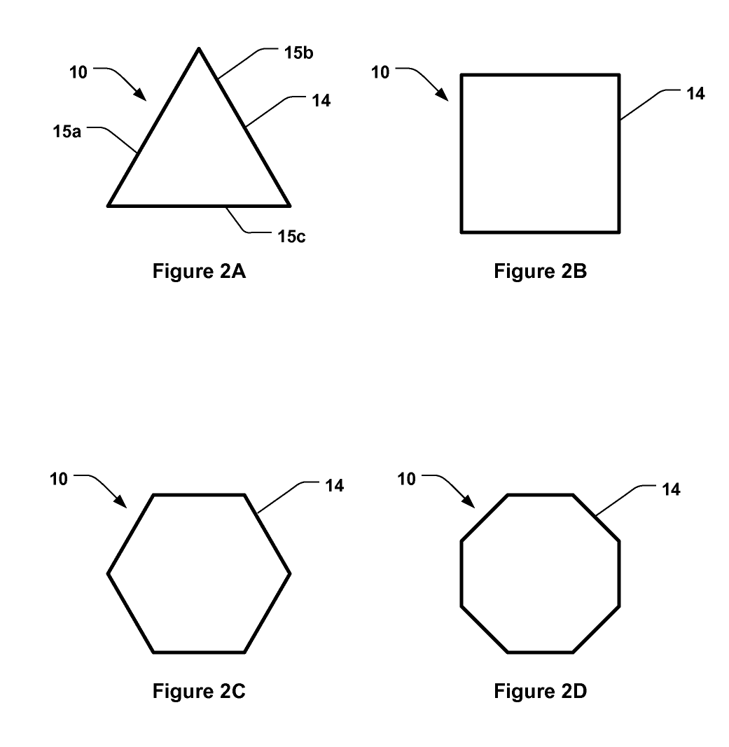

There are several alternative embodiments of the stylet within the scope of this application. Referring to FIGS. 2A-2D, the outer surface of the stylet is not circular but instead has a complex cross-sectional geometry comprising at least three faces. By way of illustration only, FIG. 2A illustrates three faces 15a-c, FIG. 2B illustrates four (non-labeled) faces, FIG. 2C illustrates six (non-labeled) faces, and FIG. 2D illustrates eight (non-labeled) faces, each taken along the view indicated as A-A in FIG. 1. As mentioned above, for simplicity only, the stylet 10 is illustrated as solid but in general it could be hollow to any degree desired.

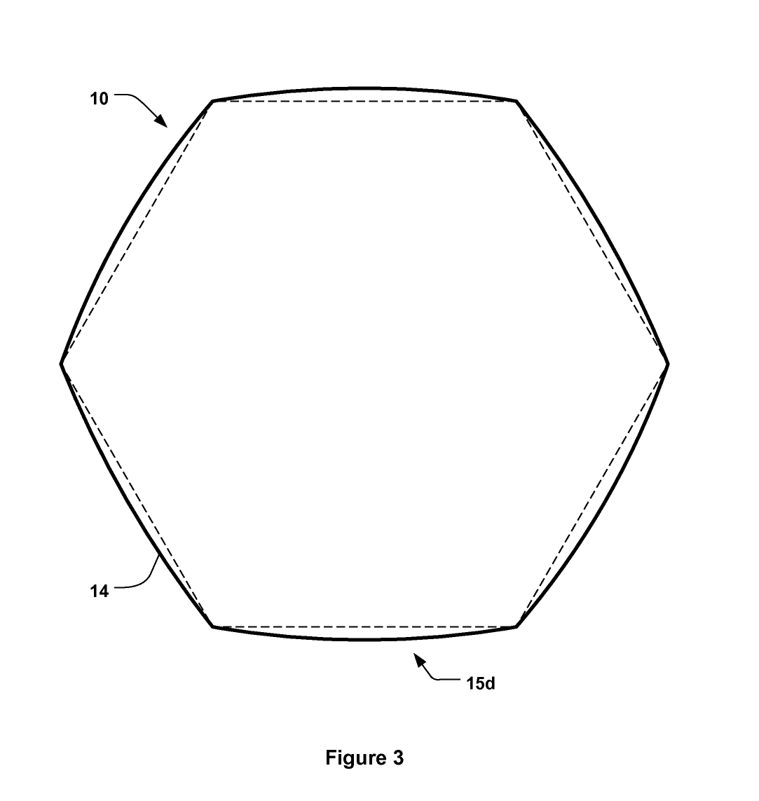

Using a six-faced configuration solely for purposes of illustration, FIG. 3 illustrates an example of a first alternative embodiment. Specifically, at least one face 15d of the outer surface 14 is concave or convex with respect to the center longitudinal axis 16 of the stylet. For purposes of illustration only, FIG. 3 depicts all six faces as convex; in general, any number of faces, from one to the maximum number present, could be convex; similarly, in general, any number of faces, from one to the maximum number present, could be concave. To illustrate the curvature of the faces illustrated in FIG. 3, the outline of a regular hexagon is illustrated in dashed lines.

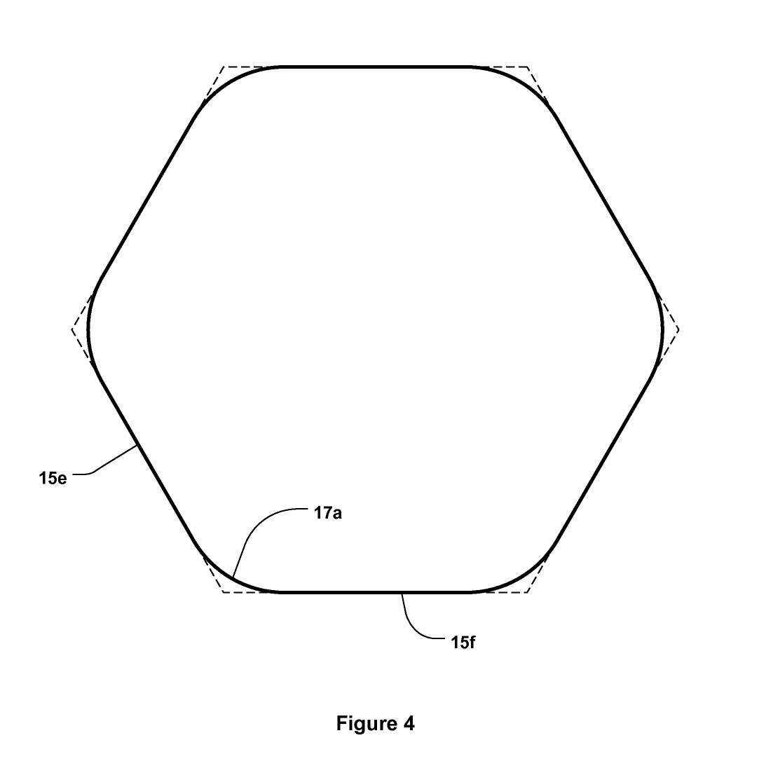

Again using a six-faced configuration solely for purposes of illustration, FIG. 4 illustrates an example of a second alternative embodiment. Specifically, a corner surface is defined as the region between immediately adjacent faces of the outer surface—for example, the region indicated as 17a between faces 15e and 15f. At least one corner surface is rounded as opposed to angular because the immediately adjacent faces have tangents (illustrated in dashed lines) which join at a point which does not lie on the corner surface. As before, for purposes of illustration only, FIG. 4 depicts all six corner surfaces as rounded, and (independently) all six are rounded to the same degree in terms of shape and size. In general, any number of them, from one to the maximum number present, could be a rounded corner surface; and each corner surface could be different from or the same as any other (although it is preferred that they all be the same as each other regardless of the shape or degree of roundness, to lend symmetry to the stylet).

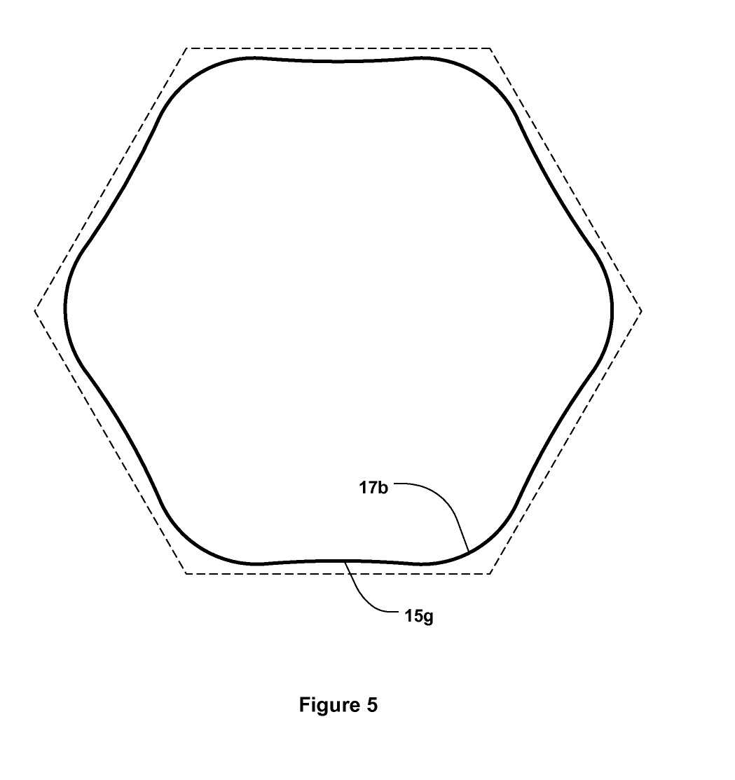

The features illustrated in FIGS. 3 and 4 could combined, e.g., a geometry could have curved faces and rounded intersections, as depicted in FIG. 5 (again using a six-faced embodiment solely as an example). In the particular example of FIG. 5, concave faces 15g (as opposed to convex faces) are illustrated as an example of the principle of combining non-straight faces with rounded corner surfaces 17b.

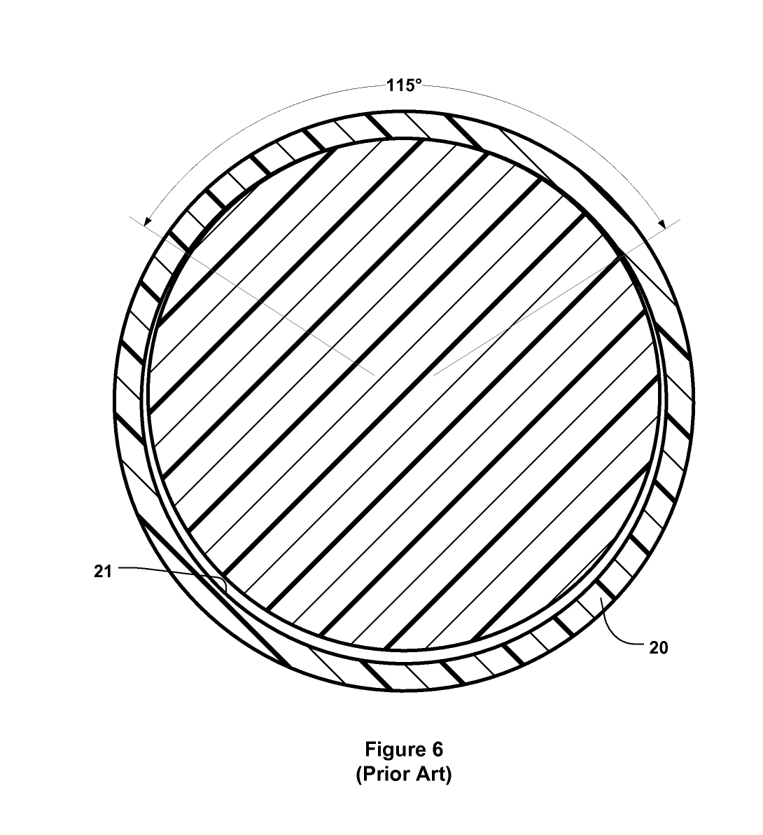

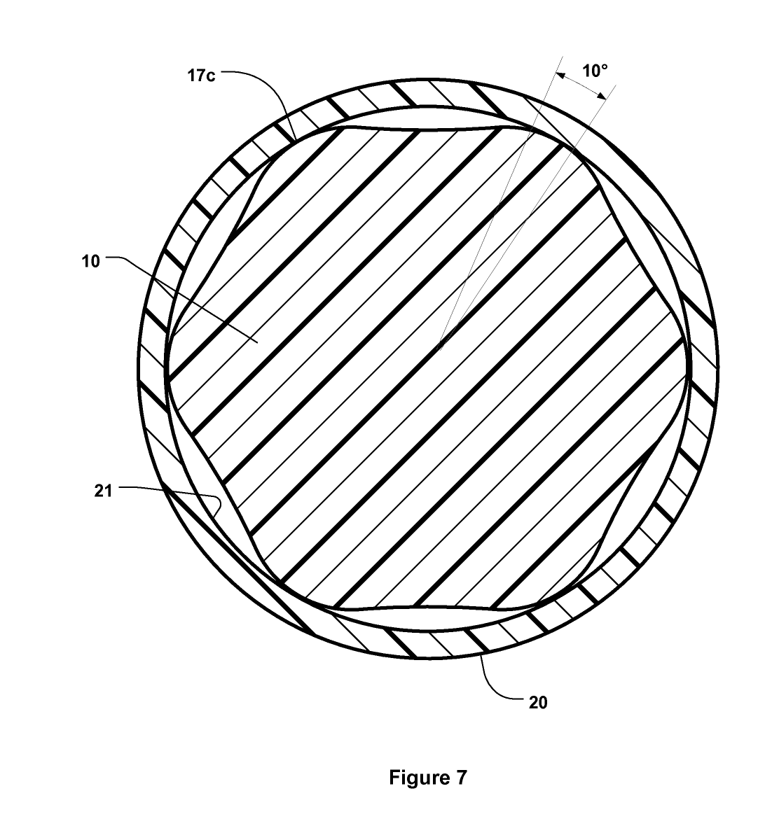

FIGS. 6 and 7 are a comparative study of the fit of a conventional round cross-section stylet (FIG. 6) and a six-faced concave-rounded embodiment (FIG. 7), each within a catheter lumen 20 which has circular inner diameter 21.

As shown in FIG. 6, the conventional circular cross-section stylet fits tightly against the inner diameter 21 of the lumen 20 over a substantial amount of arc—approximately 115 degrees, or roughly one-third of the circumference. (The exact amount will depend on the relative sizes of the stylet and lumen. In the example shown here, the stylet area is approximately 5% smaller than the area of lumen and no deflection of the inner diameter is considered.)

By comparison, the stylet of FIG. 7 intersects over a larger number of contact locations (six, corresponding to the number of rounded corner surfaces 17c), but each contact location has a small amount of contact in terms of arc—approximately 10 to 12 degrees as illustrated. Thus the total amount of contact area is only approximately 60 to 72 degrees, or approximately 50 to 65 percent as much area as the conventional fit. Because the amount of friction between the stylet and the inner diameter of the lumen depends on the amount of contact area, this is a substantial reduction.

Of course, there are potential trade-offs in terms of the amount of material in the stylet (generally proportional to the cross-sectional area) which may introduce other impacts on the “feel” or other performance of the stylet. However, in the case of many medical procedures, such as neurological procedures, the catheters and stylets are necessarily very small in cross-sectional area to begin with, and thus a relatively minor reduction in stylet cross-sectional area such as the 5% reduction described above leads to a very small reduction in amount of material (and thus a very small impact on bulk mechanical properties of the stylet). For example, in the specific case of ventricular shunt catheters, typical conventional catheter diameters have outer diameter on the order of 2.5 mm (between 7 Fr and 8 Fr) but inner diameter only on the order of 1.0 to 2.0 mm—and the stylets are necessarily smaller than the catheter inner diameter. Thus, the stylets are not very large to begin with. A reduction in stylet cross-sectional area on the order of 5% results in a very small reduction in the amount of stylet material and thus may not have an appreciable impact on “feel” and other related issues. In the particular example illustrated in FIG. 7, the cross-sectional area of the stylet is approximately 90% of the cross-sectional area of the conventional stylet of FIG. 6, but this ratio can be increased by decreasing the concavity of the faces beyond the extent shown here for clarity only.

In general, while the cross-sectional geometry could vary over the length of the stylet, it is preferred that at least for a majority of the stylet body length (and, most preferably, for essentially its entire length), the geometry remain essentially if not exactly identical.

In another embodiment, the stylet is non-circular in cross-section over its distal portion (most preferably the portion within the catheter lumen), but its proximal portion is circular in cross-section so that the “feel” of the stylet in the hand of the surgeon is not changed.

As noted before, US Published Patent Application 2008/0103448 discloses a surface treatment of a stylet which is required to have a circular cross-section, non-circular cross-sections being criticized as having unsatisfactory “feel”. In principle, such surface treatment may be applied to the surfaces of the non-circular cross-section stylets described in this application, if desired. Therefore, the entire contents of US Published Patent Application 2008/0103448 is incorporated by reference as if set forth in full. In general, that process treats, or roughens, the outer surface of the stylet body, preferably by a glass peening or a bead blasting operation, such that its maximum profile peak height is greater than 30 micrometer, its roughness average is greater than 5 micrometer, and its root-mean-square roughness is greater than 8 micrometer. More preferably, the stylet is subjected to a known peening process, in which metal or glass shot is bombarded against the surface of the stylet with suitable intensity and overlapping coverage. In the most preferred embodiment, glass shot of about 100 micrometer is used for at least 10 minutes in an intensity range between 30-60 psi. For the reasons advocated in that publication, and based on the test described there, it is desirable for the resulting treated stylet to have a removal force from a catheter of less than 0.8 lbf, more preferably about 0.1 lbf. Removal force is measured as described in that publication and the publicly available standards documents which it relies upon.

Regardless of the exact combination of structural features described above—and they have been described separately only to emphasize their independence from each other, not to imply that two or more features cannot be combined together—one preferred application of the improved stylet is in a “pre-loaded” configuration. In that configuration, the stylet is provided to the surgical site already loaded within a catheter. The primary (if not sole) function of the stylet is to provide sufficient stiffness to the catheter to assist a user in guiding the catheter to its desired location in a patient, after which the stylet is withdrawn and discarded.

Accordingly, although the invention has been described with reference to preferred embodiments, workers skilled in the art will recognize that changes can be made in form and detail without departing from the spirit and scope of the following claims.

What is claimed is:

1. In combination: a catheter made of an elastomeric material, comprising an elongate body defining within itself a lumen having a circular cross-section; and a stylet comprising an elongate stylet body having a proximal end, a distal end, and a non-circular cross-section defining an outer surface of at least three faces for at least a majority of stylet length within the lumen of the catheter.

2. The combination of claim 1, in which at least one face of the stylet outer surface is concave.

3. The combination of claim 1, in which at least one face of the stylet outer surface is convex.

4. The combination of claim 1, in which at least two immediately adjacent faces of the stylet outer surface define between themselves a rounded corner surface.

5. The combination of claim 1, in which at least one face of the stylet outer surface is concave and at least two immediately adjacent faces of the stylet outer surface define between themselves a rounded corner surface.

6. The combination of claim 1, in which at least one face of the stylet outer surface is convex and at least two immediately adjacent faces of the stylet outer surface define between themselves a rounded corner surface.

7. The combination of claim 1, in which the stylet outer surface is characterized by at least one of (a) maximum profile peak height greater than 30 micron; (b) roughness average is greater than 5 micron; and (c) root-mean-square roughness greater than 8 micron.

8. The combination of claim 1, in which the stylet is characterized by a removal force from the lumen of the catheter of less than 0.8 lbf.

9. The combination of claim 1, in which the stylet further comprises a proximal portion, outside the catheter lumen, having a circular cross-section.

10. A method of removing a stylet made of a rigid material from a catheter made of an elastomeric material to define within itself a lumen having a circular cross-section, the stylet comprising an elongate stylet body having a proximal end, a distal end, and a non-circular cross-section defining an outer surface of at least three faces at least for a majority of its length within the lumen of the catheter, the method comprising: guiding the catheter loaded with the stylet in the lumen to a desired target; and removing the stylet from the catheter.

11. The method of claim 10, in which the stylet outer surface is characterized by at least one of (a) maximum profile peak height greater than 30 micron; (b) roughness average is greater than 5 micron; and (c) root-mean-square roughness greater than 8 micron.

12. The method of claim 10, in which removing the stylet is characterized by applying to the lumen a force of less than 0.8 lbf to remove the lumen from the catheter.

13. A method of manufacturing a stylet from a rigid material, comprising providing the stylet with an elongate stylet body having a proximal end, a distal end, and a non-circular cross-section defining an outer surface of at least three faces at least for a portion of its length.

14. The method of claim 13, further comprising providing the stylet outer surface with at least one concave face.

15. The method of claim 13, further comprising providing the stylet outer surface with at least one convex face.

16. The method of claim 13, further comprising providing the stylet outer surface with at least two immediately adjacent faces which define between themselves a rounded corner surface.

17. The method of claim 13, further comprising providing the stylet outer surface with at least one concave face and at least two immediately adjacent faces of the stylet outer surface which define between themselves a rounded corner surface.

18. The method of claim 13, further comprising providing the stylet outer surface with at least one convex face and at least two immediately adjacent faces of the stylet outer surface which define between themselves a rounded corner surface.

19. The method of claim 13, further comprising providing the stylet outer surface characterized by at least one of (a) maximum profile peak height greater than 30 micron; (b) roughness average is greater than 5 micron; and (c) root-mean-square roughness greater than 8 micron.

20. The method of claim 13, further comprising providing the stylet with a proximal portion having a circular cross-section.
